(19) United States
(12) Patent Application Publication
(10) Pub. No.: US 2009/0082683 A1
(43) Pub. Date: Mar. 26, 2009

(54) Adapter device for physiological signals as well as magnetic resonance device therewith

(75) Inventors: Ernst Mustafa, Furth (NL); Michael Frank, Erlangen (DE); Sven Heggen, Erlangen (DE); Nikolaus Demharter, Dormitz (NL); Jurgen Rossler, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 12/284,590

(22) Filed: Sep. 23, 2008

(30) Foreign Application Priority Data
Sep. 25, 2007 (DE) 10 2007 045 748.2

(51) Int. Cl. A61B 5/04

(52) U.S. Cl. 600/509

(57) ABSTRACT

An adapter device for physiological signals includes a signal input for a physiological signal transformed with a measuring unit according to its measurement. The signal input connects a converter unit for retransforming the transformed physiological signal in its electrical source characteristics, which it has on the measuring unit. A signal output connected to the converter unit is provided in order to output the retransformed signal.

### CROSS REFERENCE TO RELATED APPLICATIONS

This application claims priority of German application No. 10 2007 045 748.2 filed Sep. 25, 2007, which is incorporated by reference herein in its entirety.

### FIELD OF THE INVENTION

The invention relates to an adapter device for physiological signals as well as a magnetic resonance device with an adapter device.

### BACKGROUND OF THE INVENTION

With medical imaging, physiological signals are detected when recording images of certain anatomical regions and used for synchronizing or triggering the image recording. ECG signals (electrocardiography signals) and respiration signals are detected for instance for recording the heart by means of magnetic resonance technology (MR technology) in order to synchronize the MR image recordings therewith. Here the detection including the transmission of physiological signals is only possible with specific MR-compatible devices due to the technical-physical conditions of the magnetic resonance devices. MR-compatible means that the devices used neither interfere with the operation of the MR device nor are they negatively influenced themselves by the operation of the MR device in terms of their function. It must additionally be ensured that the patient to be examined is not endangered.

It is on the other hand often necessary to continuously monitor vitally important functions of the patient, in other words before, during and after the image recording. This is carried out using patient monitoring systems, which monitor the heart function by way of ECG signals for instance and emit a signal when an abnormal status occurs.

The devices used for synchronizing and triggering the image recording are however not suitable and/or admissible as patient monitoring system. MR-compatible patient monitoring systems nevertheless exist, which may also be used as a trigger signal source for the image recording. A signal connection of the MR-compatible patient monitoring system to the MR device is however only possible by way of a special input on the MR-device and therewith introduces restrictions and a modified procedure during the preparation and examination. In particular, only the trigger signals identified by the patient monitoring system can be delivered to the MR device in a system-specific fashion. The specific trigger signal identification of the MR device can thus not be used.

Another option consists in attaching two electrode sets for the ECG detection to the patient. The first electrode set will be connected to the MR device. The ECG signals are evaluated in respect of characteristic signal responses, during the occurrence of which a trigger signal is then generated for the imaging. The second electrode set is connected to the patient monitoring system for monitoring the heart function. However, the application of two ECG electrode sets is complicated and also uncomfortable for the patient.

It is thus desirable to better integrate the patient monitoring system into the MR device.

### SUMMARY OF THE INVENTION

The object underlying the invention is now to specify an adapter device, with which a signal detection unit for physiological signals is to be used both for the trigger signal generation during the imaging as well as for a patient monitoring system. The object underlying the invention is also to specify a magnetic resonance device equipped accordingly with an adapter device.

The object is achieved by the claims. Accordingly, an adapter device for physiological signals is specified, which has a signal input for a physiological signal transformed according to its measurement with a measuring unit, a converter unit connected to the signal input for retransforming the transformed physiological signal into its electrical original properties, which it has on the measuring unit and a signal output connected to the converter unit for outputting the retransformed signal. Conventional patient monitoring systems can thus be connected to the adapter device instead of the patient and/or a signal detection unit. A flexible manufacturer-independent interface is realized for a patient monitoring system. The signal detection unit for the image recording triggering is also used for the patient monitoring system by way of the adapter device. Both the trigger function as well as the monitoring function are ensured without restrictions and without modifications to the workflow of the image recording process. The signal at the output has a level which corresponds to the measurement signal or original signal. The remaining electrical properties are also reproduced again by the retransformation, e.g. the output impedance is adjusted to the source impedance of the electrophysiological signal.

One particularly advantageous embodiment is characterized in that the signal input is embodied for a digitalized physiological signal. The interference-insensitive digital signal transmission channel for the physiological signal can thus be directly connected to the input of the adapter device. The digitalized physiological signal is again converted into an analog signal in the converter unit.

A further advantageous embodiment is characterized in that the converter unit has adjustment elements for adjusting a retransformation property. The electrical properties of the output and of the output signal and other electrical characteristics can thus be finely attuned.

With a further advantageous embodiment, a receiving unit is inserted between the signal input and the converter unit, with the receiving unit being embodied as an independent module. The receiving unit can thus be exchanged simply if a modified or also if different signal transmission systems are thus to be used between the signal detection unit and the converter unit.

A further advantageous embodiment is characterized in that a digital standard interface is present between the receiving unit and the converter unit. Standard systems can thus be used as signal transmission systems.

### BRIEF DESCRIPTION OF THE DRAWINGS

The subclaims reproduce further embodiments of the invention. Two exemplary embodiments of the invention are explained below on the basis of two Figures, in which;

FIG. 1 shows an overview representation of the design of a first adapter device for a trigger signal detection and transmission for a patient monitoring system and

FIG. 2 shows an overview representation of the design of a second adapter device for a trigger signal detection and transmission for a patient monitoring system

### DETAILED DESCRIPTION OF THE INVENTION

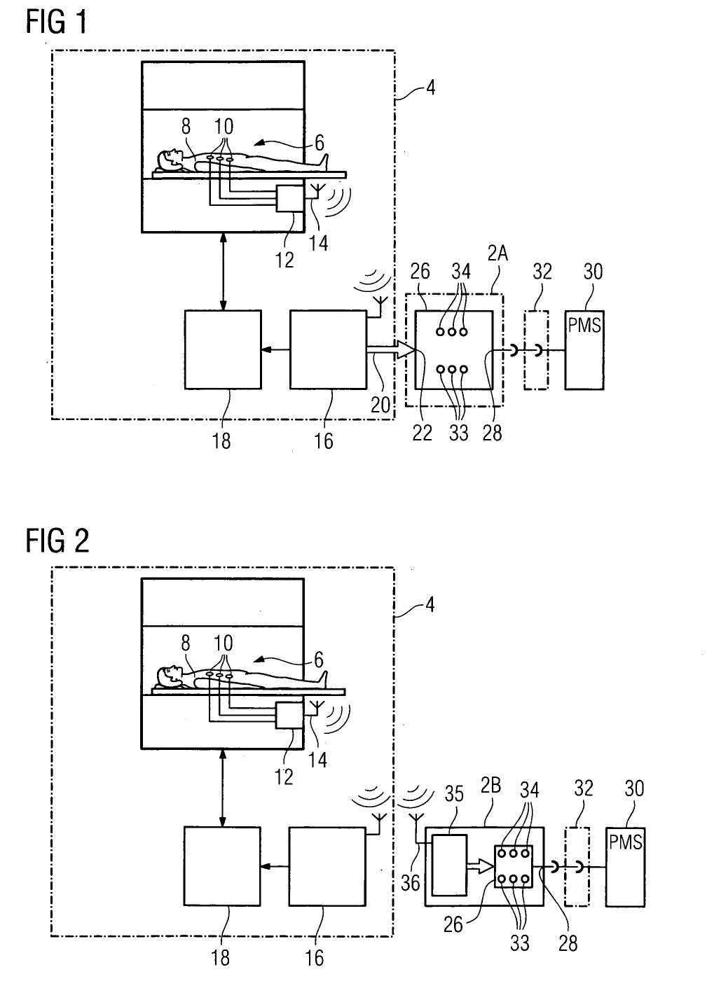

FIG. 1 shows a type of block diagram illustrating a first embodiment of an adapter device 2A for a physiological signal together with a magnetic resonance device 4. An ECG signal is measured off a patient 8 as a physiological signal using a measuring unit 6. By way of example, three ECG electrodes 10 are shown as a measuring unit 10. The ECG electrodes 10 are connected to a transmitter 12, which amplifies the ECG signal detected by the ECG electrode 10, digitalizes it and then, following a modulation, emits it by way of an antenna 14. The transmitter 12 operates in the ISM band with GFSK modulation methods and thus in a frequency range, which lies at a fail-safe distance from the operating frequency of the magnetic resonance device 4.

A trigger unit 16 for receiving and further-processing the wirelessly transmitted ECG signal forms part of the magnetic resonance device 4. The ECG signal is analyzed in the trigger unit according to characteristic signal responses, with the occurrence of which a trigger signal for the imaging being emitted to a device controller 18 of the magnetic resonance device 4.

The trigger unit 16 has a digital output 20, at which the demodulated ECG signal is output in a digital form, e.g. serially by way of a standard bus system (e.g. SPI, UART or I²C).

The output 20 is connected to a digital input 22 of the adapter device 2A. The adapter device 2A includes a converter unit 26, which regenerates a retransformed ECG signal from the digitalized ECG signal, which retransformed ECG signal has the electrical properties of the ECG signal at the ECG electrodes 10. To this end, the digitalized ECG signal is firstly converted into an analog signal by means of an analog-digital converter. A signal preparation circuit arranged downstream of the analog-digital converter then provides an ECG signal which corresponds to the originally measured ECG signal at one signal output 28 for a patient monitoring signal 30. The signal at the signal output thus has a level which corresponds to the measured ECG signal. The remaining electrical characteristics are also reproduced by the retransformation, e.g. the output impedance of the signal output 28 corresponds to the source impedance of the ECG electrodes 10 attached to the patient 8.

In order to fine-tune the electrical properties of the retransformed ECG signal at the signal output 28, adjusting elements 33 for the output level, the source impedance and if necessary other parameters, are still present on the converter unit 26.

The signal output 28 can be equipped with a conventional, standardized connector plug system (e.g. AAMI EL1-53 compatible). Furthermore, signal outputs 34 are provided, which are provided with connectors, which correspond to the connector plug system on the ECG electrodes 10 in terms of their mechanical and electrical embodiment. It is thus possible to connect the patient monitoring system 30 directly to the adapter device 2A. The connector adapter 32 allows the electrical connector of the adapter device 2A to be adjusted if necessary to different conventional connector plug systems of patient monitoring systems 30.

An integration of the adapter device 2A into the magnetic resonance device 4 is also advantageous. A special power supply can be dispensed with by the power of the adapter device 2A being supplied by way of the magnetic resonance device. Furthermore, the signal line connection from the output 20 to the digital input 22 is then dispensed with by way of a freely moveable and openly positioned signal cable. “Cable spaghetti” at this point is thus avoided.

FIG. 2 likewise shows a block diagram of a further embodiment of an adapter device 2B. In contrast to the embodiment according to FIG. 1, the electrical connection 20 with the device controller 18 is dispensed with here. Instead, the adapter device 2B includes a receiving unit 35 with an antenna 36 as a signal input. The receiving unit 35 is embodied as an exchangeable module. The simple exchange allows the adapter device 2B to be adjusted to different wireless transmission channels. The receiving unit 35 demodulates the received transformed ECG signal into a digital signal, like the trigger unit 16 already described previously on the basis of FIG. 1.

The digital signal is then fed to the converter unit 26. The function of the converter unit 26 as well as the connection with the patient monitoring system is already described on the basis of FIG. 1.

1.-13. (canceled)

14. An adapter device for a physiological signal of a patient during a medical imaging, comprising:
a measuring unit that measures the physiological signal;
a signal input that inputs the physiological signal transformed with the measuring unit based on the measurement;
a converter unit connected to the signal input that retransforms the transformed physiological signal into a retransformed signal having an electrical original property on the measuring unit; and
a signal output connected to the converter unit that outputs the retransformed signal.

15. The adapter device as claimed in claim 14, wherein the transformed physiological signal is a digitalized physiological signal.

16. The adapter device as claimed in claim 14, wherein the transformed physiological signal is a modulated signal.

17. The adapter device as claimed in claim 14, wherein the converter unit comprises an adjusting element that adjusts a retransformation property.

18. The adapter device as claimed in claim 14, wherein the physiological signal is an ECG signal and the retransformed signal has a property of the ECG signal on an ECG electrode.

19. The adapter device as claimed in claim 14, wherein the signal output is embodied accordingly like the signal input on an ECG electrode.

20. The adapter device as claimed in claim 14, wherein the signal output is a standard plug system.

21. The adapter device as claimed in claim 14, wherein the signal input is an antenna that wirelessly receives the transformed physiological signal.

22. The adapter device as claimed in claim 14, wherein a receiving unit is inserted between the signal input and the converter unit.

23. The adapter device as claimed in claim 22, wherein the receiving unit is an independent module.

24. The adapter device as claimed in claim 22, wherein a digital interface is provided between the receiving unit and the converter unit.

25. The adapter device as claimed in claim 14, wherein the signal output comprises an electrical connector.

26. The adapter device as claimed in claim 25, wherein the electrical connector is adjusted to different connector plug systems of a patient monitoring system connected to the adapter device via a plug adapter.

27. A magnetic resonance device, comprising:
an integrated adapter device comprising:
a measuring unit that measures a physiological signal of a patient during a medical imaging by the magnetic resonance device;
a signal input that inputs the physiological signal transformed with the measuring unit based on the measurement;
a converter unit connected to the signal input that retransforms the transformed physiological signal into a retransformed signal having an electrical original property on the measuring unit; and
a signal output connected to the converter unit that outputs the retransformed signal.
